(19) United States
(12) Patent Application Publication
(10) Pub. No.: US 2008/0200803 A1
(43) Pub. Date: Aug. 21, 2008

(54) Cancer Detection and Treatment Instrument

(75) Inventors: Young Woo Kwon, Seoul (KR); Chang Yul Cheon, Seoul (KR); Jei Won Cho, Gyeonggi-do (KR); Jeong Hoon Yoon, Seoul (KR)

Correspondence Address: CANTOR COLBURN, LLP, 20 Church Street, 22nd Floor, Hartford, CT, 06103, US

(73) Assignee: SEOUL NATIONAL UNIVERSITY INDUSTRY FOUNDATION, Seoul (KR)

(21) Appl. No.: 11/718,823

(22) PCT Filed: Nov. 1, 2006
(86) PCT No.: PCT/KR2006/004507

§ 371 Date: May 8, 2007

(30) Foreign Application Priority Data
Nov. 8, 2005 (KR) 1020050106661

(51) Int. Cl. A61B 18/18; A61B 5/05; B23P 15/00

(52) U.S. Cl. 600/430; 606/33; 29/527.1

(57) ABSTRACT

Provided herein is a cancer detection and treatment instrument comprising: a first conductive plate; a second conductive plate which is opposed to the first conductive plate and has a first opening; a first signal line disposed between the first conductive plate and the second conductive plate; a first contact member of which one end is exposed through the first opening and of which the other end is connected to the first signal line; a dielectric portion filled between the first and second conductive plates and the first signal line; and a conductive layer surrounding both side surfaces and a front end surface of the dielectric portion which are exposed. Therefore, it is possible to accurately detect cancer by the use of the ultrahigh-frequency signal and to treat a diseased portion without damaging tissues around the diseased portion.

### TECHNICAL FIELD

The present invention relates to a cancer detection and treatment instrument, and more particularly, to an instrument for detecting and treating cancer using an ultrahigh-frequency wave.

### BACKGROUND ART

In general, cancer is detected using a variety of methods. For example, a mammography method is used to detect mammary cancer. However, the detection of mammary cancer using the method depends upon ages of patients or structures of mammary tissues. It is easy to detect a problem in the mammary tissues using the method, but it is dangerous to judge the abnormal lesion as cancer. Therefore, the same detection test should be repeated or other tests such as ultrasonic diagnosis or biopsy should be used.

The ultrasonic diagnosis is effective only when cancer cells have a size larger than a predetermined size and have a morphological variation. The biopsy can cause de-formation of cell shapes and has a difficulty in judging metastasis of cancer cells. The above-mentioned methods are only means for detecting cancer, and thus an additional method for treating cancer should be considered.

### DISCLOSURE OF INVENTION

### Technical Problem

The present invention is contrived to solve the above-mentioned problem. An advantage of the present invention is that it provides a cancer detection and treatment instrument capable of accurately detecting cancer by the use of ultrahigh-frequency waves and treating a diseased portion without damaging tissues around the diseased portion.

### Technical Solution

According to an aspect of the invention, there is provided a cancer detection and treatment instrument comprising: a first conductive plate; a second conductive plate which is opposed to the first conductive plate and has a first opening; a first signal line disposed between the first conductive plate and the second conductive plate; a first contact member of which one end is exposed through the first opening and of which the other end is connected to the first signal line; a dielectric portion filled between the first and second conductive plates and the first signal line; and a conductive layer surrounding both side surfaces and a front end surface of the dielectric portion which are exposed.

The cancer detection and treatment instrument may further comprise: a second opening formed in the second conductive plate; a second signal line disposed between the first conductive plate and the second conductive plate; a second contact member of which one end is exposed through the second opening and of which the other end is connected to the second signal line; and a ground plate disposed between the first signal line and the second signal line.

The dielectric portion and the first and second conductive plates may have a sharp front end.

The opening may have a circular shape.

One end of the first signal line may be exposed externally.

The cancer detection and treatment instrument may further comprise: a monolithic microwave integrated circuit supplying a detection-specific ultrahigh-frequency signal and a detection-specific ultrahigh-frequency power to the first signal line and receiving reflected signals through the first signal line; and a digital signal processor receiving the reflected signals from the monolithic microwave integrated circuit, analyzing electromagnetic characteristics of the reflected signals, and controlling a treatment-specific ultrahigh-frequency power in accordance with the analysis result.

The conductive layer and the first contact member may be made of conductive epoxy.

In addition, the cancer detection and treatment instrument may further comprise: a second opening formed in the first conductive plate; a second signal line disposed between the first conductive plate and the second conductive plate; a second contact member of which one end is exposed through the second opening and of which the other end is connected to the second signal line; and a ground plate disposed between the first signal line and the second signal line.

On the other hand, the cancer detection and treatment instrument may further comprise: a second opening formed in the first conductive plate; a second signal line which is disposed between the first conductive plate and the second conductive plate and of which one end extends as long as the first and second conductive plates and is exposed externally; a third signal line disposed between the second signal line and the second conductive plate; a second contact member of which one end is exposed through the second opening and of which the other end is connected to the third signal line; a first ground plate disposed between the first signal line and the second signal line; and a second ground plate disposed between the second signal line and the third signal line.

According to another aspect of the invention, there is provided a method of manufacturing a cancer detection and treatment instrument. The method comprises: disposing a first dielectric member between first and second metal plates and bonding the first metal plate, the first dielectric member, and the second metal plates; etching the first metal plate by the use of a photolithography technique to form a signal line; bonding a second dielectric member and a third metal plate to each other; forming an opening in the third metal plate; forming a through-hole penetrating the second dielectric member at the center of the opening; bonding the second dielectric member to the signal line; filling the through-hole with conductive epoxy; and coating both side surfaces and a front end surface of the first and second dielectric members with conductive epoxy.

According to another aspect of the invention, there is provided a method of manufacturing a cancer detection and treatment instrument. The method comprises: bonding a first metal plate, a first dielectric member, a second metal plate, a second dielectric member, and a third metal plate in this order; etching the first and third metal plates by the use of a photolithography technique to form a first signal line and a second signal line, respectively; bonding a fourth metal plate and a third dielectric member to each other; bonding a fifth metal plate and a fourth dielectric member to each other; forming a first opening and a second opening in the fourth metal plate and the fifth metal plate, respectively; forming a first through-hole and a second through-hole penetrating the third dielectric member and the fourth dielectric member at the centers of the first opening and the second opening, respectively; bonding the third dielectric member and the fourth dielectric member onto the first signal line and the second signal line, respectively; filling the first through-hole and the second through-hole with conductive epoxy; and coating both side surfaces and a front end surface of the first to fourth dielectric members with conductive epoxy.

According to another aspect of the invention, there is provided a method of manufacturing a cancer detection and treatment instrument. The method comprises: bonding a first metal plate, a first dielectric member, a second metal plate, a second dielectric member, and a third metal plate in this order; etching the first and third metal plates by the use of a photolithography technique to form a first signal line and a second signal line, respectively; bonding a third dielectric member, a fourth metal plate, a fourth dielectric member, and a fifth metal plate in this order; etching the fifth metal plate by the use of a photolithography technique to form a third signal line; bonding a sixth metal plate and a fifth dielectric member to each other; bonding a seventh metal plate and a sixth dielectric member; forming a first opening and a second opening in the sixth metal plate and the seventh metal plate, respectively; forming a first through-hole and a second through-hole penetrating the fifth dielectric member and the sixth dielectric member at the centers of the first opening and the second opening, respectively; bonding the third dielectric member onto the second signal line; bonding the fifth dielectric member and the sixth dielectric member onto the first signal line and the third signal line, respectively; filling the first through-hole and the second through-hole with conductive epoxy; and coating both side surfaces of the first to sixth dielectric members with conductive epoxy. Here, the second signal line extends as long as the third dielectric member and is exposed externally.

According to another aspect of the invention, there is provided a cancer detection and treatment instrument comprising: a first electronic device; a second electronic device connected to the first electronic device; and a probe unit connected to the first electronic device so as to come in contact with a human tissue. Here, the first electronic device includes a radio frequency (RF) signal generator, a switching unit, a scattering parameter measuring unit connected to the RF signal generator and the switching unit, and a power amplifier. The second electronic device includes a signal characteristic extractor and a controller.

The RF signal generator, the switching unit, the scattering parameter measuring unit, and the power amplifier may be integrated, and the signal characteristic extractor and the controller may be integrated.

The first electronic device may further include an incident and reflected power measuring unit and a power distribution unit connected to the switching unit and the power amplifier.

In addition, the incident and reflected power measuring unit and the power distribution unit may be integrated.

The cancer detection and treatment instrument may further comprise an impedance tuning unit.

In addition, the impedance tuning unit may be integrated.

The probe unit may supply a detection-specific signal and a treatment-specific signal to a diseased portion.

The frequency of the detection-specific signal may be determined in accordance with a microwave characteristic of the diseased portion.

The frequency of the treatment-specific signal may be determined in accordance with a microwave characteristic of the diseased portion.

The probe unit may supply an RF signal to the diseased portion from the first electronic device.

The frequency of the RF signal generated from the RF signal generator may be in the range of 5 to 60 GHz.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram schematically illustrating a cancer detection and treatment instrument according to an exemplary embodiment of the invention;

FIG. 2 is a plan view illustrating a probe unit of the cancer detection and treatment instrument according to an exemplary embodiment of the invention;

FIG. 3 is a cross-sectional view taken along Line III-III of FIG. 2;

FIG. 4 is a plan view illustrating a probe unit of an cancer detection and treatment instrument according to another exemplary embodiment of the invention;

FIG. 5 is a cross-sectional view illustrating a probe unit of a cancer detection and treatment instrument according to another exemplary embodiment of the invention;

FIG. 6 is a cross-sectional view taken along Line VI-VI of FIG. 5;

FIGS. 7 to 14 are diagrams sequentially illustrating a method of manufacturing the probe unit shown in FIG. 2;

FIG. 15 is an exploded perspective view illustrating a probe unit of a cancer detection and treatment instrument according to another exemplary embodiment of the invention;

FIGS. 16 to 21 are diagrams sequentially illustrating a method of manufacturing the probe unit shown in FIG. 8;

FIG. 22 is an exploded perspective view illustrating a probe unit of a cancer detection and treatment instrument according to another exemplary embodiment of the invention; and

FIG. 23 is a diagram partially illustrating a method of manufacturing the probe unit shown in FIG. 10.

### BEST MODE FOR CARRYING OUT THE INVENTION

Hereinafter, exemplary embodiments of the present invention will be described in detail with reference to the attached drawings so that those skilled in the art can easily put the invention into practice. However, the invention may be embodied in a variety of forms, but is not limited to the exemplary embodiments.

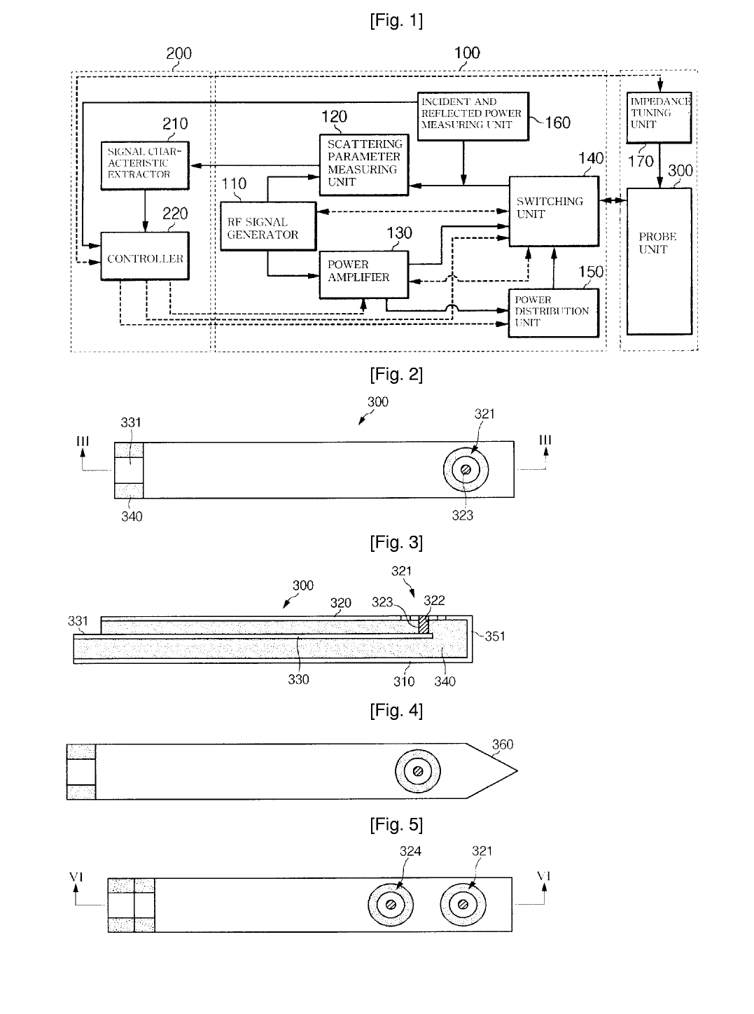

FIG. 1 is a diagram schematically illustrating a cancer detection and treatment instrument according to an exemplary embodiment of the invention.

Referring to FIG. 1, the cancer detection and treatment instrument according to the exemplary embodiment includes a first electronic device 100, a second electronic device 200, and a probe unit 300.

The first electronic device 100 transmits a ultrahigh-frequency signal to the probe unit 300 and receives an ultrahigh-frequency signal from the probe unit 300. The first electronic device 100 measures the magnitude and phase of the received ultrahigh-frequency signal and delivers the measured data to the second electronic device 200. The first electronic device 100 supplies ultrahigh-frequency power for treating a diseased portion to the probe unit 300.

The first electronic device 100 includes a radio frequency (RF) signal generator 110, a scattering parameter measuring unit 120, a power amplifier 130, a switching unit 140, and a power distribution unit 150. The constituent elements of the first electronic device 100 may be integrated to form a monolithic microwave integrated circuit.

The RF signal generator 110 generates RF waves having a specific frequency which are delivered to the diseased portion through the probe unit 300 for use in detection or treatment.

The scattering parameter measuring unit 120 measures an incident signal and a reflected signal by receiving the signal which is emitted from the RF signal generator 110 and reflected by the diseased portion.

The power amplifier 130 amplifies the signal generated from the RF signal generator 110 so as to be suitable for the detection or the treatment of the diseased portion.

The switching unit 140 switches a cancer detection system and a cancer treatment system and performs the switching so that the RF signal can be delivered or measured in several directions when the probe unit 300 operates in the several directions.

The power distribution unit 150 serves to divide and supply the ultrahigh-frequency power into the several directions when the detection or treatment is performed in the several directions by the use of the probe unit 300.

The cancer detection and treatment instrument according to the exemplary embodiment of the invention may further include an incident and reflected power measuring unit 160 and an impedance tuning unit 170. Microwaves are reflected by portions having different impedance. Accordingly, the generation of a reflected signal reduces the magnitude of the delivered signal, thereby deteriorating the efficiency. Therefore, the impedance tuning unit 170 matches the impedance of the diseased portion with the impedance of the cancer detection and treatment instrument. That is, the impedance tuning unit 170 minimizes the reflected power at the time of treatment by measuring the reflected power and matching the impedance with each other while varying the impedance.

The second electronic device 200 is connected to the first electronic device 100, and serves to receive measured data from the first electronic device 100 and to extract and analysis characteristic parameters such as dielectric constant. The second electronic device 200 also controls the treatment-specific ultrahigh-frequency power corresponding to the data.

The second electronic device 200 includes a signal characteristic extractor 210 and a controller 220. The signal characteristic extractor 210 and the controller 220 are preferably integrated to form a monolithic chip of a digital signal processor.

The signal characteristic extractor 210 serves to a unique parameter indicating a microwave characteristic of a material.

The controller 220 controls the constituent units 110, 120, 130, and 140 of the first electronic device 100 and the signal characteristic extractor 210.

The probe unit 300 is inserted directly into the diseased portion. The probe unit 300 delivers the ultrahigh-frequency signal from the first electronic device 100 to the diseased portion and delivers the reflected signal reflected by the diseased portion to the first electronic device 100, thereby detecting the state of the diseased portion. The probe unit 300 directly delivers the power from the first electronic device 100 to the diseased portion, thereby treating the diseased portion. That is, the probe unit 300 performs the detection and treatment by delivering the detection-specific signal or the treatment-specific signal from the first electronic device 100 to the diseased portion. The frequencies of the detection-specific signal and the treatment-specific signal are determined depending upon the microwave characteristic of the diseased portion. The frequency of the RF signal delivered from the RF signal generator 110 of the first electronic device 100 to the diseased portion through the probe unit 300 is preferably in the range of 5 to 60 GHz.

As a result, it is possible to simultaneously detect and treat the diseased portion by the use of the RF, to more accurately detect the diseased portion, and to more effectively treat the diseased portion.

FIG. 2 is a plan view illustrating the probe unit 300 of the cancer detection and treatment instrument according to the exemplary embodiment of the invention, and FIG. 3 is a cross-sectional view taken along Line III-III of FIG. 2 and illustrates the cancer detection and treatment instrument.

Referring to FIGS. 2 and 3, the probe unit 300 of the cancer detection and treatment instrument according to the exemplary embodiment includes a first conductive plate 310, a second conductive plate 320 opposed to the first conductive plate 310, a signal line 330 formed between the first and second conductive plates 310 and 320, and a dielectric portion 340 filled between the first and second conductive plates 310 and 320 and the signal line 330.

The first and second conductive plates 310 and 320 are made of a conductor and have a sufficient length to be directly inserted into the diseased portion. The insertion of the probe unit 300 can be easily performed using a guide cannular or the like. An opening 321 is formed in the second conductive plate 320. The opening 321 is a portion coming in direct contact with the diseased portion to perform the cancer detection and treatment. The opening 321 has a circular shape. The opening 321 may have a variety of shapes such as a quadrangular shape, depending upon the purposes of the detection and treatment.

A through-hole 322 penetrating the dielectric portion 340 is formed at the center of the opening 321. The through-hole 322 is filled with a contact member 323. One end of the contact member 323 is exposed from the opening 321 and the other end is connected to the signal line 330. The contact member 323 may be made of conductive epoxy.

The signal line 330 serves to deliver a signal or power and is formed in the longitudinal direction of the first and second conductive plates 310 and 320. One end of the signal line 330 is connected to the opening 321 through the contact member 323. The other end of the signal line 330 is exposed externally to form a pad portion 331 for connection to the first electronic device 100.

The dielectric portion 340 fills between the first and second conductive plates 310 and 320 and the signal line 330 and is made of a general insulating material.

On the other hand, a conductive layer is formed on both side surfaces and the front end surface 351 of the dielectric portion 340. The conductive layer serves to help with the safe deliver of a signal of the signal line 330 and may be made of conductive epoxy.

The front end 360 of the probe unit may be formed sharp for the purpose of easy insertion into the diseased portion as shown in FIG. 4.

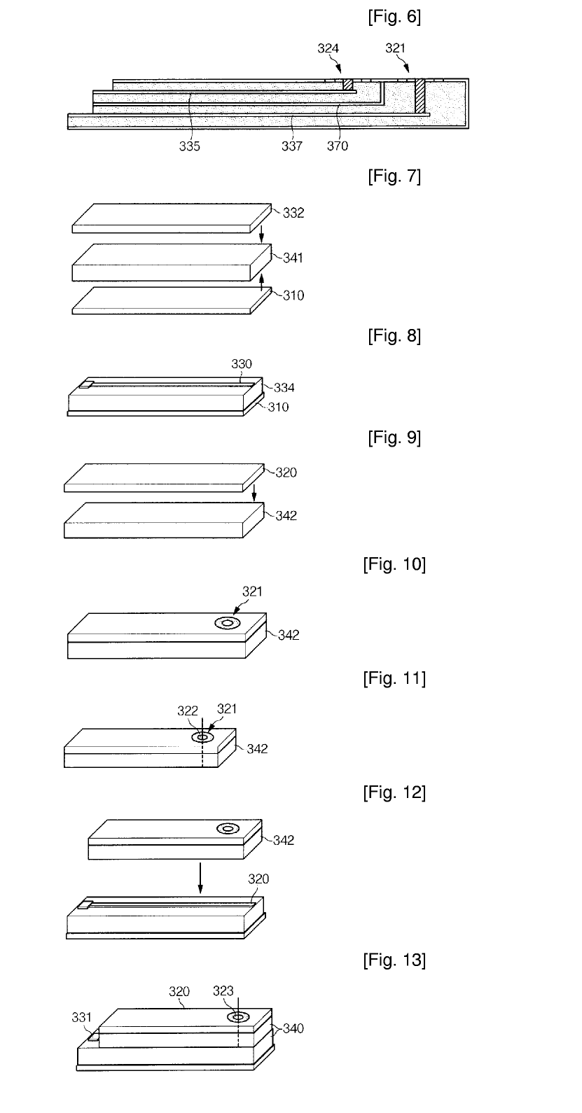

In the probe unit 300, two or more openings 321 and 324 may be formed in the second conductive plate 320 as shown in FIGS. 5 and 6. Accordingly, two or more signal lines 335 and 337 may be connected to the openings 321 and 324, respectively. In this case, a ground plate 370 is formed between both signal lines 335 and 337.

On the other hand, the probe unit may be used for the detection and treatment of cancer along with a vector circuit analyzer, a commercial power amplifier, and other ultrahigh-frequency circuit devices instead of the first and second electronic devices 100 and 200.

Operations of the cancer detection and treatment instrument having the above-mentioned structure will be described.

The probe unit 300 is inserted into a portion considered as a diseased portion, and an ultrahigh-frequency signal is irradiated to the portion. Then, a reflected signal is generated in response to the irradiated ultrahigh frequency signal, is received by the probe unit 300, and then is delivered to the first electronic device 100. A diseased portion and a normal portion have electromagnetic characteristics different from each other in response to a ultrahigh-frequency wave. Accordingly, the first and second electronic devices 100 and 200 analyze the electromagnetic characteristics such as a dielectric constant of the reflected signal to determine whether the diseased portion is infected. Thereafter, the time and frequency of the treatment-specific ultrahigh-frequency power are determined depending upon the determined state of the diseased portion. Then, the probe unit 300 supplies the determined treatment-specific ultrahigh-frequency signal to the diseased portion. The ultrahigh-frequency signal locally heats only the diseased portion to treat the diseased portion. After the treatment, it is possible to confirm the treated state by checking the ultrahigh-frequency characteristic of the diseased portion.

A method of manufacturing the probe unit of the cancer detection and treatment instrument will be described now.

FIGS. 7 to 14 are diagrams sequentially illustrating a method of a probe unit according to an exemplary embodiment of the invention.

Referring to FIG. 7, a first dielectric member 341 is interposed between first and second metal plates 332 and 310 and then is bonded thereto. Thereafter, the first metal plate 332 is etched by the use of a photolithography technique as sown in FIG. 8 to form the signal line 330.

Subsequently, as shown in FIG. 9, a second dielectric member 342 and a third metal plate 320 which becomes the second conductive plate are bonded to each other. As shown in FIG. 10, the opening 321 is formed in the third metal plate 320. The opening 321 can be formed by etching the metal plate into a ring shape by the use of the photolithography technique. The through-hole 322 penetrating the second dielectric member 342 is formed at the center of the opening 321 as shown in FIG. 11.

Thereafter, as shown in FIG. 12, the second dielectric member 342 and the signal line 320 are bonded to each other. At this time, the lengths of the second dielectric member 342 and the third metal plate 320 are set smaller than the length of the signal line 320, thereby externally exposing the signal line 320 to form the pad portion 331 for connection to the first electronic device 100. Then, as shown in FIG. 13, the contact member 323 is formed by filling the through-hole 322 with, for example, conductive epoxy and then heating and curing the conductive epoxy. The contact member 323 connects one end of the signal line 320 to the opening 321.

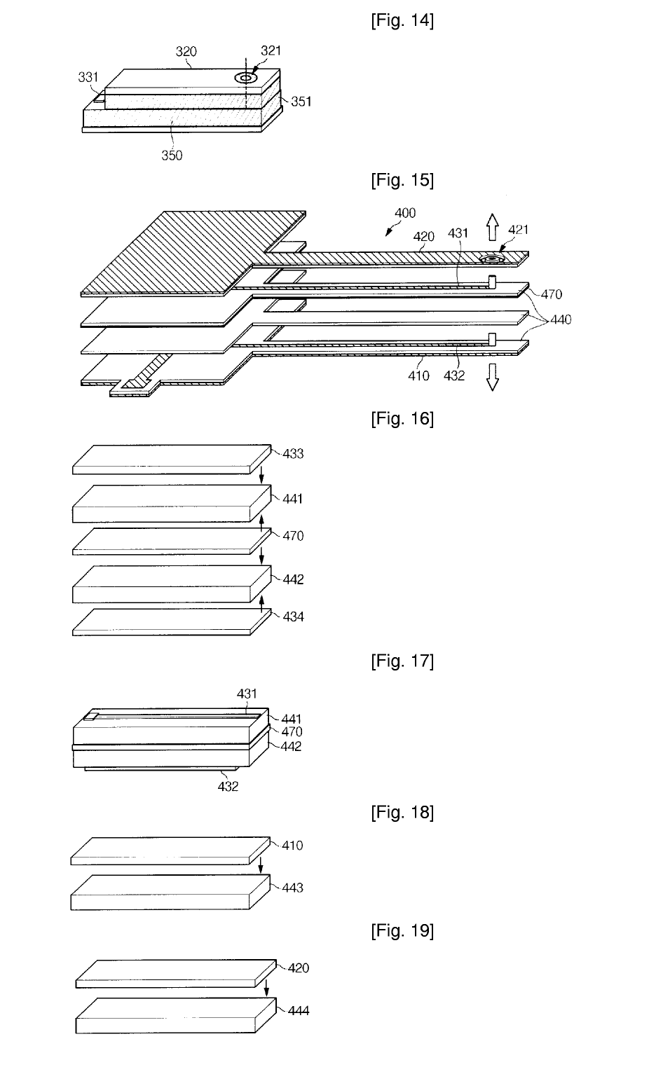

Finally, as shown in FIG. 14, a conductive layer is formed on both exposed side surfaces 350 and the front end surface 351 of the dielectric portion 340 including the first and second dielectric members 341 and 342. The conductive layer may be made of conductive epoxy.

FIG. 15 is an exploded perspective view illustrating a probe unit 400 according to a cancer detection and treatment instrument according to another exemplary embodiment of the invention. A structure different from the probe unit 300 shown in FIG. 2 is mainly described.

Referring to FIG. 15, the probe unit 400 according to the exemplary embodiment includes a first opening (not shown) and a second opening 421 formed in first and second conductive plates 410 and 420, respectively. First and second signal lines 431 and 432 are formed to correspond to the first and second openings 421. A ground plate 470 serving as a ground is formed between the first and second signal lines 431 and 432. Since the openings are formed in the top and bottom conductive plates 410 and 420, it is possible to perform the cancer detection and treatment of the diseased portion in two directions.

FIGS. 16 to 21 are diagrams illustrating a method of manufacturing the probe unit 400 shown in FIG. 15.

Referring to FIG. 16, a first metal plate 433, a first dielectric member 441, a second metal plate 470, a second dielectric member 442, and a third metal plate 434 are sequentially stacked and bonded. Thereafter, as shown in FIG. 17, the first and third metal plates 433 and 434 are etched using a photolithography technique to form first and second signal lines 431 and 432. Here, the second metal plate 470 serves as a ground plate.

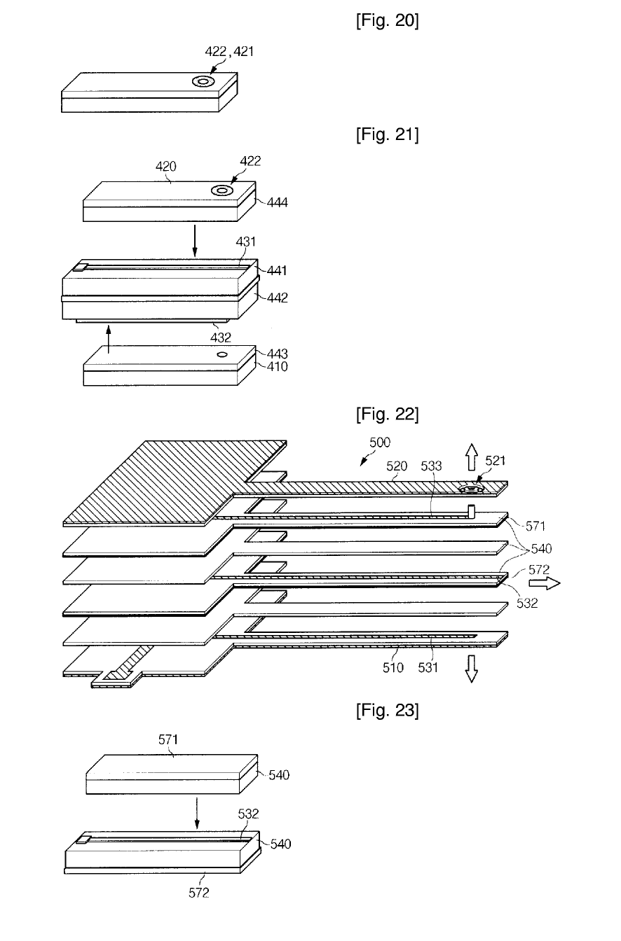

As shown in FIG. 18, a fourth metal plate 410 and a third dielectric member 443 are bonded to each other, and as shown in FIG. 19, a fifth metal plate 420 and a fourth dielectric member 444 are bonded to each other. Thereafter, as shown in FIG. 20, first and second openings 421 and 422 are formed in the fourth and fifth metal plates 410 and 420, respectively. The openings 421 and 422 can be formed by etching the fourth and fifth metal plates 410 and 420 by the use of a photolithography technique to remove parts of the metal plates into a ring shape. Thereafter, through-holes penetrating the third and fourth dielectric members 443 and 444, respectively, are formed at the center of the respective openings 421 and 422.

Subsequently, as shown in FIG. 21, the fourth dielectric member 444 is bonded to the first signal line 431 and the third dielectric member 443 is bonded to the second signal line 432. Thereafter, contact members are formed by filling the through-holes with conductive epoxy and heating and curing the conductive epoxy.

Finally, a conductive layer (not shown) is formed on both side surfaces and the front end surface of the first to fourth dielectric members 441 to 444, wherein the surfaces are exposed. The conductive layer may be made of conductive epoxy.

FIG. 22 is an exploded perspective view illustrating a probe unit 500 of a cancer detection and treatment instrument according to another exemplary embodiment of the invention. A structure different from the probe unit 400 shown in FIG. 15 and the probe unit 300 shown in FIG. 2 is mainly described now.

Referring to FIG. 22, in the probe unit 500 according to the exemplary embodiment, a first opening (not shown) and a second opening 521 are formed in first and second conductive plates 510 and 520, respectively. Accordingly, a first signal line 531 connected to the first opening and a third signal line 533 connected to the second opening 521 are formed. A second signal line 532 is formed between the first and third signal lines 531 and 533. One end of the second signal line 532 extends up to the end of the metal plates or dielectric members and is exposed externally. Accordingly, the cancer detection and treatment of the probe unit 500 can be performed in three directions of an upward direction, a downward direction, and a forward direction. On the other hand, a first ground plate 572 is disposed between the first and second signal lines 531 and 532 and a second ground plate 571 is disposed between the second and third signal lines 532 and 533.

The probe unit 500 shown in FIG. 22 can be manufactured by forming a structure including two ground plates 571 and 572, the second signal line 532 formed between both ground plates 571 and 572, and a dielectric member 540 filled between both ground plates 571 and 572 and the second signal line 532, instead of the ground plate 470 in the method of manufacturing the probe unit 400 shown in FIG. 15. At this time, the second signal line 532 extends as long as the dielectric member 540 or both ground plates 571 and 572 and is exposed externally. When the second signal line 532 does not have a length enough to be exposed externally, the second signal line 532 can be exposed externally by cutting the manufactured probe unit.

### INDUSTRIAL APPLICABILITY

According to the invention described above, it is possible to more accurately detect cancer by the use of ultrahigh-frequency waves and to treat the diseased portion by the use of a heating remedy without damaging tissues around the diseased portion.

Although the exemplary embodiments of the invention have been described in detail, the invention is not limited to the exemplary embodiments, but it will be understood by those skilled in the art that various modifications, additions and substitutions are possible without departing from the scope and spirit of the invention as disclosed in the accompanying claims.

1. A cancer detection and treatment instrument comprising:
a first conductive plate;
a second conductive plate which is opposed to the first conductive plate and has a first opening;
a first signal line disposed between the first conductive plate and the second conductive plate;
a first contact member of which one end is exposed through the first opening and of which the other end is connected to the first signal line;
a dielectric portion filled between the first and second conductive plates and the first signal line; and
a conductive layer surrounding both side surfaces and a front end surface of the dielectric portion, the surfaces being exposed.

2. The cancer detection and treatment instrument according to claim 1, further comprising:
a second opening formed in the second conductive plate;
a second signal line disposed between the first conductive plate and the second conductive plate;
a second contact member of which one end is exposed through the second opening and of which the other end is connected to the second signal line; and
a ground plate disposed between the first signal line and the second signal line.

3. The cancer detection and treatment instrument according to claim 1, wherein the dielectric portion and the first and second conductive plates have a sharp front end.

4. The cancer detection and treatment instrument according to claim 1, wherein the opening has a circular shape.

5. The cancer detection and treatment instrument according to claim 1, wherein one end of the first signal line is exposed externally.

6. The cancer detection and treatment instrument according to claim 1, further comprising:
a monolithic microwave integrated circuit supplying a detection-specific ultrahigh-frequency signal and a detection-specific ultrahigh-frequency power to the first signal line and receiving reflected signals through the first signal line; and
a digital signal processor receiving the reflected signals from the monolithic microwave integrated circuit, analyzing electromagnetic characteristics of the reflected signals, and controlling a treatment-specific ultrahigh-frequency power in accordance with the analysis result.

7. The cancer detection and treatment instrument according to claim 1, wherein the conductive layer and the first contact member are made of conductive epoxy.

8. The cancer detection and treatment instrument according to claim 1, further comprising:
a second opening formed in the first conductive plate;
a second signal line disposed between the first conductive plate and the second conductive plate;
a second contact member of which one end is exposed through the second opening and of which the other end is connected to the second signal line; and
a ground plate disposed between the first signal line and the second signal line.

9. The cancer detection and treatment instrument according to claim 1, further comprising:
a second opening formed in the first conductive plate;
a second signal line which is disposed between the first conductive plate and the second conductive plate and of which one end extends as long as the first and second conductive plates and is exposed externally;
a third signal line disposed between the second signal line and the second conductive plate;
a second contact member of which one end is exposed through the second opening and of which the other end is connected to the third signal line;
a first ground plate disposed between the first signal line and the second signal line; and
a second ground plate disposed between the second signal line and the third signal line.

10. A method of manufacturing a cancer detection and treatment instrument, the method comprising:
disposing a first dielectric member between first and second metal plates and bonding the first metal plate, the first dielectric member, and the second metal plates;
etching the first metal plate by the use of a photolithography technique to form a signal line;
bonding a second dielectric member and a third metal plate to each other;
forming an opening in the third metal plate;
forming a through-hole penetrating the second dielectric member at the center of the opening;
bonding the second dielectric member to the signal line;
filling the through-hole with conductive epoxy; and
coating both side surfaces and a front end surface of the first and second dielectric members with conductive epoxy.

11. A method of manufacturing a cancer detection and treatment instrument, the method comprising:
bonding a first metal plate, a first dielectric member, a second metal plate, a second dielectric member, and a third metal plate in this order;
etching the first and third metal plates by the use of a photolithography technique to form a first signal line and a second signal line, respectively;
bonding a fourth metal plate and a third dielectric member to each other;
bonding a fifth metal plate and a fourth dielectric member to each other;
forming a first opening and a second opening in the fourth metal plate and the fifth metal plate, respectively;
forming a first through-hole and a second through-hole penetrating the third dielectric member and the fourth dielectric member at the centers of the first opening and the second opening, respectively;
bonding the third dielectric member and the fourth dielectric member onto the first signal line and the second signal line, respectively;
filling the first through-hole and the second through-hole with conductive epoxy; and
coating both side surfaces and a front end surface of the first to fourth dielectric members with conductive epoxy.

12. A method of manufacturing a cancer detection and treatment instrument, the method comprising:
bonding a first metal plate, a first dielectric member, a second metal plate, a second dielectric member, and a third metal plate in this order;
etching the first and third metal plates by the use of a photolithography technique to form a first signal line and a second signal line, respectively;
bonding a third dielectric member, a fourth metal plate, a fourth dielectric member, and a fifth metal plate in this order;
etching the fifth metal plate by the use of a photolithography technique to form a third signal line;
bonding a sixth metal plate and a fifth dielectric member to each other;
bonding a seventh metal plate and a sixth dielectric member;
forming a first opening and a second opening in the sixth metal plate and the seventh metal plate, respectively;
forming a first through-hole and a second through-hole penetrating the fifth dielectric member and the sixth dielectric member at the centers of the first opening and the second opening, respectively;
bonding the third dielectric member onto the second signal line;
bonding the fifth dielectric member and the sixth dielectric member onto the first signal line and the third signal line, respectively;
filling the first through-hole and the second through-hole with conductive epoxy; and
coating both side surfaces of the first to sixth dielectric members with conductive epoxy,
wherein the second signal line extends as long as the third dielectric member and is exposed externally.

13. A cancer detection and treatment instrument comprising:
a first electronic device;
a second electronic device connected to the first electronic device; and
a probe unit connected to the first electronic device so as to come in contact with a human tissue,
wherein the first electronic device includes a radio frequency (RF) signal generator, a switching unit, a scattering parameter measuring unit connected to the RF signal generator and the switching unit, and a power amplifier, wherein the second electronic device includes a signal characteristic extractor and a controller.

14. The cancer detection and treatment instrument according to claim 13, wherein the RF signal generator, the switching unit, the scattering parameter measuring unit, and the power amplifier are integrated, and the signal characteristic extractor and the controller are integrated.

15. The cancer detection and treatment instrument according to claim 13, wherein the first electronic device further includes an incident and reflected power measuring unit and a power distribution unit connected to the switching unit and the power amplifier.

16. The cancer detection and treatment instrument according to claim 15, wherein the incident and reflected power measuring unit and the power distribution unit are integrated.

17. The cancer detection and treatment instrument according to claim 13, further comprising an impedance tuning unit.

18. The cancer detection and treatment instrument according to claim 17, wherein the impedance tuning unit is integrated.

19. The cancer detection and treatment instrument according to claim 13, wherein the probe unit supplies a detection-specific signal and a treatment-specific signal to a diseased portion.

20. The cancer detection and treatment instrument according to claim 19, wherein the frequency of the detection-specific signal is determined in accordance with a microwave characteristic of the diseased portion.

21. The cancer detection and treatment instrument according to claim 19, wherein the frequency of the treatment-specific signal is determined in accordance with a microwave characteristic of the diseased portion.

22. The cancer detection and treatment instrument according to claim 19, wherein the probe unit supplies an RF signal to the diseased portion from the first electronic device.

23. The cancer detection and treatment instrument according to claim 13, wherein the frequency of the RF signal generated from the RF signal generator is in the range of 5 to 60 GHz.
